(19) United States
(12) Patent Application Publication
(10) Pub. No.: US 2015/0361018 A1
(43) Pub. Date: Dec. 17, 2015

(54) PROCESS FOR CLEANING CONTAMINATED FLUIDS

(71) Applicant: Evan Koslow, Dallas, TX (US)

(72) Inventor: Evan Koslow, Dallas, TX (US)

(21) Appl. No.: 14/740,995

(22) Filed: Jun. 16, 2015

Related U.S. Application Data
(60) Provisional application No. 62/012,840, filed on Jun. 16, 2014.

(51) Int. Cl. C07C 41/40; B01D 21/01

(52) U.S. Cl. CPC C07C 41/40; B01D 21/01

(57) ABSTRACT

A process for cleaning contaminants from a contaminated fluid includes a) combining the contaminated fluid with a less polar miscible solvent to yield a reduced polarity contaminated fluid-solvent mixture in which the contaminants are insoluble, at least a portion of the contaminants precipitating from the reduced polarity contaminated fluid-solvent mixture to yield precipitated contaminants and the reduced polarity contaminated fluid-solvent mixture; b) separating at least a portion of the precipitated contaminants from the reduced polarity contaminated fluid-solvent mixture; and c) separating at least a portion of the fluid from the reduced polarity contaminated fluid-solvent mixture to yield cleaned fluid.

### FIELD

The disclosure relates to contaminated fluids, such as glycol that has been fouled during use in a glycol dehydrating process. More specifically, the disclosure relates to processes for cleaning contaminated glycol via the precipitation of contaminants from the contaminated glycol.

### INTRODUCTION

Glycol dehydrating processes are widely used for the reduction of moisture in natural gas and preparation of this gas for pipeline distribution. Triethylene glycol (TEG) is the most common fluid used within these processes. In these processes, raw natural gas is passed through a bubble-tray contacting tower in a counter-current direction to a flow of TEG. This is accomplished at a relatively low temperature, where the moisture is rapidly adsorbed into the liquid.

The wet TEG emerging from the process is sent to a boiler where the fluid is heated to drive off the moisture. The dry fluid is then cooled in a heat exchanger and usually filtered using a combination of a particulate filter and an activated carbon filter. This filtration aims to remove contaminants that accumulate in the glycol, such as higher hydrocarbons, particulates, and a wide variety of higher molecular weight polar contaminants (HMWPC).

HMWPC are usually composed of a complex mixture of polar carboxylic acids, polycarboxylic acids, fatty acids, cross-linked carboxylic acids, and polymerized polycarboxylic acids. Because many of these are charged molecules and associated with counter-ions, they contribute to the accumulation of salt within the glycol.

One of the core problems in glycol dehydrating processes is that the current filtration technologies poorly control the accumulation of HMWPC. The accumulating HMWPC are entirely stable and fully dispersed within the glycol, and particularly in triethylene glycol due to its polar nature, and tend to pass through ordinary particulate filters. In addition, the polar nature of HMWPC discourages adsorption onto activated carbon, and even if adsorbed, they are likely to be displaced from the surface of the activated carbon by other nonpolar higher hydrocarbons.

Under these conditions, the HMWPC continue to accumulate regardless of the use of various filters, and eventually foul the entire system. The HMWPC then build up and lead to a large increase in viscosity of the fluid, are the major cause of foaming within the bubble tray tower, interfere with the absorption process, and eventually force the replacement of the fluid within the system.

There are roughly 36,000 of these glycol-dehydrating processes operating in the United States, and an even greater number throughout the world. Overall, there may be over 100,000 of these systems of various sizes operating worldwide.

### SUMMARY OF SOME EMBODIMENTS

The following summary is intended to introduce the reader to various aspects of the applicant's teaching.

According to one aspect, a process for cleaning contaminants from a contaminated fluid comprises a) combining the contaminated fluid with a less polar miscible solvent to yield a reduced polarity contaminated fluid-solvent mixture in which the contaminants are insoluble. At least a portion of the contaminants precipitate from the reduced polarity contaminated fluid-solvent mixture to yield precipitated contaminants and the reduced polarity contaminated fluid-solvent mixture. The process further comprises b) separating at least a portion of the precipitated contaminants from the reduced polarity contaminated fluid-solvent mixture, and c) separating at least a portion of the fluid from the reduced polarity contaminated fluid-solvent mixture to yield cleaned glycol.

According to a further embodiment, the process further comprises, prior to combining the contaminated fluid with the less polar miscible solvent, combining hydrogen peroxide with the contaminated fluid and heating the hydrogen peroxide-contaminated fluid mixture.

According to another aspect, a process for precipitating contaminants from contaminated glycol comprises combining the contaminated glycol with a less polar miscible solvent to yield a reduced polarity glycol-solvent mixture in which the contaminants are insoluble. At least a portion of the contaminants precipitate from the reduced polarity glycol-solvent mixture to yield precipitated contaminants and the reduced polarity glycol-solvent mixture.

According to another aspect, a less polar miscible solvent is used to precipitate contaminants from contaminated glycol. The less polar miscible solvent is combined with the contaminated glycol to yield a reduced polarity solvent in which the contaminants are insoluble.

The contaminated fluid may comprise an amine solution or a glycol. The glycol may comprise a glycol selected from the group consisting of triethylene glycol, diethylene glycol, ethylene glycol, tetraethylene glycol, and combinations thereof. In one specific example, the contaminated glycol comprises contaminated triethylene glycol.

The less polar miscible solvent may comprise an alkyl acetate. The alkyl acetate may be selected from the group consisting of methyl acetate, ethyl acetate, propyl acetate, isopropyl acetate, butyl acetate, and combinations thereof. In one specific example, the less polar miscible solvent comprises a mixture of propyl acetate and isopropyl acetate. The less polar miscible solvent may comprise a generally equal amount of propyl acetate and isopropyl acetate by volume.

The processes may be any one of a continuous process, a semi-continuous process, and a batch process.

Step b) may comprise filtering the precipitated contaminants from the reduced polarity glycol-solvent mixture.

The contaminants may comprise high molecular weight polar contaminants. The high molecular weight polar contaminants may be selected from the group consisting of polar carboxylic acids, polycarboxylic acids, fatty acids, cross-linked carboxylic acids, and polymerized polycarboxylic acids.

Step c) may comprise evaporating the less polar miscible solvent from the reduced polarity glycol-solvent mixture. The process may further comprise condensing the less polar miscible solvent and recycling the less polar miscible solvent to step a).

The contaminated glycol and the less polar miscible solvent may be combined at a volumetric ratio of between about 1:0.5 and about 1:5.

According to another aspect, a process for cleaning high molecular weight polar contaminants from contaminated triethylene glycol comprises a) combining the contaminated triethylene glycol with an alkyl acetate solvent to yield a reduced polarity triethylene glycol-alkyl acetate mixture in which the contaminants are insoluble. At least a portion of the high molecular weight polar contaminants precipitate from the reduced polarity triethylene glycol-alkyl acetate mixture to yield precipitated high molecular weight polar contaminants and the reduced polarity triethylene glycol-alkyl acetate mixture. The process further comprises b) separating at least a portion of the precipitated high molecular weight polar contaminants from the reduced polarity triethylene glycol-alkyl acetate mixture; and c) separating at least a portion of the triethylene glycol from the reduced polarity triethylene glycol-alkyl acetate mixture.

According to another aspect, a process for cleaning contaminants from contaminated glycol includes a) combining hydrogen peroxide with the contaminated glycol, and b) heating the hydrogen peroxide-glycol mixture such that the hydrogen peroxide is decomposed, to yield a hydrogen peroxide-glycol mixture with reduced optical density.

The hydrogen peroxide combined with the glycol may be between about 0.1% to about 6% concentration by weight.

The hydrogen peroxide-glycol mixture may be heated to between about 120 degrees C. to about 170 degrees C.

According to another aspect, a process for cleaning contaminants from a contaminated non-polar substance includes combining the contaminated non-polar substance with a more polar miscible solvent to yield an increased polarity non-polar substance-solvent mixture in which the contaminants are insoluble, at least a portion of the contaminants precipitating from the increased polarity non-polar substance-solvent mixture to yield precipitated contaminants and the increased polarity non-polar substance-solvent mixture.

The non-polar substance may be contaminated lube oil.

The contaminants may comprise high molecular weight polar contaminants.

### BRIEF DESCRIPTION OF THE DRAWINGS

The drawings included herewith are for illustrating various examples of articles, methods, and apparatuses of the present specification and is not intended to limit the scope of what is taught in any way. In the drawings:

FIG. 1 is a process flow diagram of an example process for cleaning contaminants from contaminated glycol.

FIG. 2 is a photograph of samples having the addition of water to glycol.

FIG. 3 is a photograph of glycol and hydrogen peroxide samples after treatment.

FIG. 4 is a photograph of filtrates and filters where a hydrogen peroxide treatment process has been used.

FIG. 5 is a chart of percent reduction in optical density versus amount of applied hydrogen peroxide in treatment process.

FIG. 6 is a chart of percent reduction in contaminate versus the acetate to glycol ratio.

### DETAILED DESCRIPTION OF SOME EMBODIMENTS

Various apparatuses or processes will be described below to provide an example of an embodiment of the claimed subject matter. No embodiment described below limits any claim and any claim may cover processes or apparatuses that differ from those described below. The claims are not limited to apparatuses or processes having all of the features of any one apparatus or process described below or to features common to multiple or all of the apparatuses described below. It is possible that an apparatus or process described below is not an embodiment of any exclusive right granted by issuance of this patent application. Any apparatus or process described below and for which an exclusive right is not granted by issuance of this patent application may be the subject matter of another protective instrument, for example, a continuing patent application, and the applicants, inventors or owners do not intend to abandon, disclaim or dedicate to the public any such apparatus or process by its disclosure in this document.

Contaminants may be cleaned from contaminated glycol by combining the contaminated glycol with a less polar miscible solvent. Combining the contaminated glycol with a less polar miscible solvent yields a mixture of reduced polarity (otherwise referred to herein as a “reduced polarity mixture” or a “reduced polarity glycol-solvent mixture”), in which the contaminants are generally insoluble. At least a portion of the contaminants therefore precipitate from the reduced polarity mixture, to yield precipitated contaminants in solid form. The precipitated contaminants may then be separated from the reduced polarity mixture. Furthermore, the glycol may then be separated from the reduced polarity mixture.

The contaminated glycol may be any type of glycol, including but not limited to triethylene glycol, diethylene glycol, ethylene glycol, tetraethylene glycol, and combinations thereof. In one particular example, the contaminated glycol may be triethylene glycol.

The less polar miscible solvent may be any solvent that is miscible with the glycol, but that is less polar than the glycol. The solvent may be as non-polar as possible, while still being miscible with the glycol.

In some examples, the less polar miscible solvent may be an alkyl acetate. For example, the alkyl acetate may be methyl acetate, ethyl acetate, propyl acetate, isopropyl acetate, butyl acetate, or combinations thereof.

In one particular example, the less polar miscible solvent is a mixture of propyl acetate and isopropyl acetate. The mixture of propyl acetate and isopropyl acetate may include, for example, about a 1:3 volumetric ratio of propyl acetate to isopropyl acetate, a 3:1 volumetric ratio of propyl acetate to isopropyl acetate, or generally equal amounts of propyl acetate and isopropyl acetate by volume (i.e. approximately a 1:1 volumetric ratio of propyl acetate to isopropyl acetate).

In other examples, other less polar miscible solvents may be used.

The contaminants may in some examples be high molecular weight polar contaminants, such as polar carboxylic acids, polycarboxylic acids, fatty acids, cross-linked carboxylic acids, polymerized polycarboxylic acids, and combinations thereof. In other examples, other contaminants may alternatively or additionally be present.

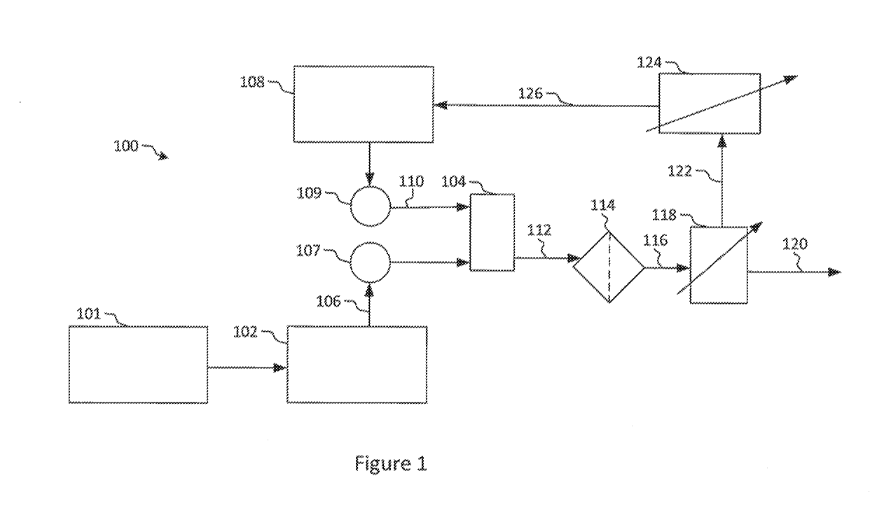

Referring now to FIG. 1, an example of a process 100 for cleaning contaminants from contaminated fluid, such as glycol is shown. The process 100 is a continuous process. However, in alternative examples, the process may be a batch or semi-continuous process.

In the example shown, contaminated glycol is fed from a contaminated glycol tank 102 to a mixer 104 via line 106. Line 106 may include a flow-meter 107. Less polar miscible solvent is fed from a solvent tank 108 to the mixer 104 via line 110. Line 110 may include a flow-meter 109.

In the mixer 104 the contaminated glycol is combined with the less polar miscible solvent to yield a reduced polarity mixture.

The ratio of contaminated glycol and less polar miscible solvent may be controlled with the use of variable-speed pumps, throttling valves, or other controls. In some examples, the contaminated glycol and the less polar miscible solvent may be combined at a volumetric ratio of between about 1:0.5 and 1:5, more specifically between about 1:2 and about 1:4. In one specific example, the contaminated glycol and the less polar miscible solvent may be combined at a volumetric ratio of about 1:3.5.

As mentioned above, the reduced polarity mixture is generally incapable of maintaining the contaminants in solution, and at least a portion of the contaminants therefore precipitate from the reduced polarity mixture.

The precipitated contaminants and reduced polarity mixture are then fed via line 112 to a solids separator 114, where at least a portion of the precipitated contaminants are separated from the reduced polarity mixture. The solids separator 114 may in some examples be a filter, such as a particulate filter, for filtering the precipitated contaminants from the reduced polarity mixture. In other examples, another type of solids separator may be used, such as a centrifuge.

Optionally, an additional solids separator (not shown) may be provided downstream of the solids separator 114. For example, an activated carbon filter may be provided downstream of the solids separator 114.

The reduced polarity mixture, now without the precipitated contaminants, may be fed via line 116 to a liquid separator 118, where at least a portion of the glycol is separated from the reduced polarity mixture to yield cleaned glycol. The liquid separator 118 may in some examples be an evaporator which evaporates the less polar miscible solvent, leaving the cleaned glycol in liquid form. The cleaned glycol may leave the liquid separator 118 via line 120.

The cleaned glycol may then optionally be reused, for example in a glycol dehydration process.

In some examples, a slip-stream of cleaned glycol may be fed back to the mixer (not shown).

The evaporated less polar miscible solvent may leave the evaporator via line 122 as a vapor, and may be fed to a condenser 124 where it is condensed back to a liquid state. The liquid less polar miscible solvent may then optionally be recycled back to the solvent tank 108 via line 126.

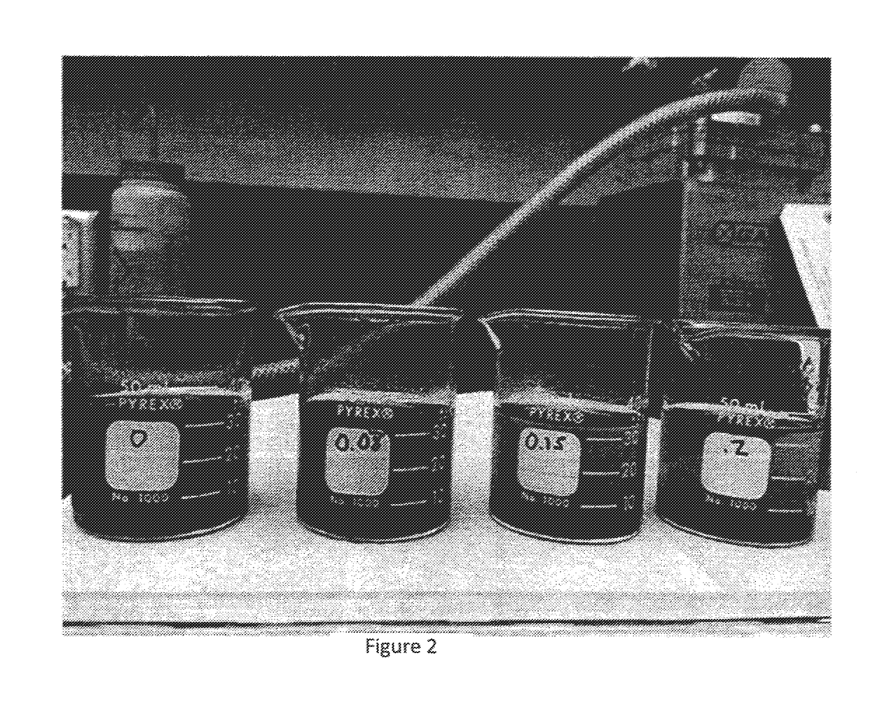

In an embodiment, the contaminated glycol may be treated with hydrogen peroxide, at 101 and as described with reference to FIGS. 2-6. The treatment may include combining a desired amount of hydrogen peroxide to the contaminated glycol, for example, as described with reference to Experiments F and G below. The hydrogen peroxide and contaminated glycol mixture may be heated, for example, as described with reference to Experiment F below. In an embodiment, the hydrogen peroxide combined with the glycol may have an effective range of about 0.1% to 6% concentration by weight.

Treatment with hydrogen peroxide may include heating the hydrogen peroxide and contaminated glycol mixture to the point where the decomposition of the hydrogen peroxide is observed by the generation of oxygen bubbles and where the residual water is boiled. Such decomposition and boiling may take place in the temperature range of between about 100 degrees C. to about 170 degrees C. In a further embodiment, the hydrogen peroxide and contaminated glycol mixture is heated to between about 120 degrees C. to about 170 degrees C.

In an embodiment, hydrogen peroxide is used to reduce the contaminant without combining with the less polar miscible solvent to clean contaminants from the contaminated glycol. Optical density may be reduced after the hydrogen peroxide treatment even prior to further treatment with acetate.

In an embodiment, treatment of the sample by heating with a small amount of hydrogen peroxide (3% by weight) may provide deep cleaning of the contaminant with small amounts of acetate. A 10-fold reduction in acetate may be possible while continuing to achieve 87% contamination reduction in a single pass. Reduction of acetate by 5-fold may allow for contamination reduction by 93% in a single pass. For example, with a cost of the hydrogen peroxide at about $0.06/gallon glycol treated, it may cost about $120 to process a 2,000-gallon glycol volume.

The processes described herein may be used to clean contaminants from substances other than glycol. For example, the processes described herein may be used to clean contaminants from other polar substances. In such examples, a less polar miscible solvent may be combined with the contaminated polar substance to yield a reduced polarity mixture in which the contaminants are insoluble. At least a portion of the contaminants may then precipitate. In one particular example, the polar substance may be a contaminated amine solution, and contaminants may be cleaned from the contaminated amine solution by combining the contaminated amine solution with a less polar miscible solvent.

In other examples, the processes described herein may be used to clean contaminants from non-polar substances. In such examples, a more polar miscible solvent may be combined with the contaminated non-polar substance to yield an increased polarity mixture in which the contaminants are insoluble. At least a portion of the contaminants may then precipitate. In one particular example, the non-polar substance may be contaminated lube oil, and contaminants may be cleaned from contaminated lube oil by combining the contaminated lube oil with a more polar miscible solvent to yield an increased polarity mixture.

In an embodiment, there may be trace water in some of the samples that causes a separate phase to form when mixed with acetate. Such trace water may be reduced in samples obtained from the lean-side of the glycol process. Hydrogen peroxide treatment may serve to reduce this moisture, as the sample is heated and dried during the hydrogen peroxide treatment.

In an embodiment, the acetate causes a precipitate to form. This precipitation reaction may occur rapidly at high contamination levels and high applied acetate concentrations. The reaction may slow for samples containing reduced amounts of contamination and when using smaller amounts of acetate. The precipitate may be composed of large particles that are visible to the eye after 20-30 minutes. An inexpensive filter may be used to separate this precipitate. The precipitated particles may remain fine when low concentrations of acetate are used. If there is a low acetate injection, a good filter may be used.

In an embodiment, samples respond in varying degrees to the hydrogen peroxide treatment and acetate cleaning. A laboratory method may include methodical instructions to an operator on how to program the machine. In certain cases, the hydrogen peroxide treatment may not be carried out aggressively as a polymerization reaction is followed by a oxidation reaction which could make the contaminant difficult to remove. In this case, small amounts of hydrogen peroxide are used.

In an embodiment, the reduction of acetate used may allow for: i) an increase in the amount of glycol processed during each pass through the reactor, ii) a decrease in the volume of acetate that is to be evaporated and recycled, iii) an improvement in processing time and reduction in energy consumption, iv) an overall improvement in process throughput of 2.3-fold by volume and approximately 4-fold by boiler processing time, for a total improvement of 9.3-fold, i.e. more than 9-times more throughput for a given reactor size running at a fixed energy input, and v) reduced acetate consumption as a result of reduced losses during recycling.

In an embodiment, the machine is only slightly more complex with the hydrogen peroxide treatment, however the total reactor size may be reduced while sustaining or increasing the original throughput while the operating cost may be reduced. In an embodiment, the system may have a throughput of about 3 gallons/hour and a reactor size of about 15 gallons.

### EXAMPLES

### Rapid Test for Cleaning of HMWPC from Glycol

Approximately 3 ml of contaminated glycol was combined with approximately 15 ml of a candidate less polar miscible solvent (also referred to as a “candidate solvent”) in a 125 ml Erlenmeyer flask. The tested candidate solvents are listed in Table 1. The fluids were swirled to cause mixing, and gentle swirling was continued for 15 seconds followed by settling for approximately 1 minute. At the end of this 1 minute period, the mixture was poured into a Whatman GF-C filter cup mounted on a vacuum flask. A mild vacuum was applied to cause the filtrate to be collected in a large test tube. The filter was a light grey when the HMWPC failed to precipitate, but was a dark black color and oily when the HMWPC precipitated and was captured by the filter. Using this simple qualitative test, it was observed that each of the candidate solvents cause precipitation of HMWPC when mixed at a volumetric ratio of 5:1 glycol:candidate solvent, and that the filtrate was a light tea-colored material in comparison to the original contaminated glycol, which is a thick black liquor.

### Quantitative Assessment of Various Candidate Solvents to Precipitate Contaminants

The performance of the candidate solvents to cause the precipitation of HMWPC from contaminated glycol was assessed by measuring the optical clarity (at a wavelength of 620 nm) of the cleaned glycol after filtration by a standard filter.

Contaminated glycol was first mixed with varying quantities of triethylene glycol (TEG). In all cases, the amount of contaminant within the system was held constant by using a constant amount of the contaminated glycol. To this mixture of dirty glycol (DG) and diluent TEG was added a variable quantity of candidate solvent, such that the combined amount of candidate solvent and diluent TEG was constant. Hence, the concentration of dark-colored contaminated glycol in the sample, and the amount of clear fluids (diluent TEG or clean solvent) were both held constant. In this manner, the original (prior to cleaning) optical density of each sample was held constant, but the ratio of test chemical to total TEG was varied.

The diluent glycol and contaminated glycol were first mixed and then the candidate solvent was added, gently mixed by swirling for 30 seconds, and then subjected to vacuum filtration through a standard Whatman GF/C filter funnel.

The filtrate was collected and placed into a glass cuvette and inserted into a spectrophotometer set to measure the absorbance of light at 620 nm. Results are shown in Table 1. The limit of the spectrophotometer's sensitivity is an absorbance of 3.5. Prior to each sample being measured, the spectrophotometer was zeroed using a sample of clean analytical-grade TEG. The optical density of the original contaminated glycol solution was too high to be measurable. However, an 11-fold dilution with clean TEG had an absorbance of 2.613, which implies that the original sample had an absorbance of 28.74. Because each volume of contaminated glycol was mixed with five-volumes of combined diluent TEG and candidate solvent, the optical density of the filtrate was diluted by six-fold. The optical density (absorbance) of a sample that appears to have been entirely cleared of HMWPC was 0.336. This is believed to be the result of other colored contaminants within the fluid that are not removed by this type of solvent cleaning.

Absorbance is related to contaminant concentration through the Beer-Lambert equation:

A=εbc (1)

Where A is absorbance, ε is the molar absorptivity of light within the sample with units of L mol⁻¹ cm⁻¹, b is the path length of the sample (i.e. the path length of the cuvette in which the sample is contained), and c is the concentration of the compound in solution. This equation can be normalized as simple ratios between the absorptivity of the original contaminated glycol (Aₒ) to the absorptivity of the cleaned glycol (Aₛₐₘₚₗₑ) while subtracting the absorptivity of the non-HMWPC contaminants (A_{background}) to calculate the fraction of contamination remaining (Fᵣ):

Fᵣ=6*[(Aₛₐₘₚₗₑ−A_{background})/(Aₒ)]=0.208*(Aₛₐₘₚₗₑ−0.336) (2)

and %Reduction=(1−Fᵣ)*100 (3)

Based upon this equation and the 3.5-absorbance limit of the spectrometer, it can be seen that that less than 65% of the original HMWPC contamination can remain within the clarified sample to produce a filtrate of sufficient optical clarity for the spectrometer to measure.

TABLE 1
ASSESSING CHEMICALS FOR CLEANING DIRTY GLYCOL
(volumes in ml)

| Volume of | Volume of | Volume of | | | |
|---|---|---|---|---|---|
| Contam. | Candidate | Diluent | CS/ | | |
| Glycol (CG) | Solvent (CS) | TEG | (CG + TEG) | Aₛₐₘₚₗₑ | Fᵣ |
| | methyl acetate | | | | |
| 3 | 14 | 1 | 3.50 | >3.5 | >0.65 |
| 3 | 15 | 0 | 5.00 | 0.905 | 0.118 |
| | ethyl acetate | | | | |
| 3 | 13 | 2 | 2.60 | 3.143 | 0.584 |
| 3 | 14 | 1 | 3.50 | 2.160 | 0.379 |
| 3 | 15 | 0 | 5.00 | 0.454 | 0.024 |
| | propyl acetate | | | | |
| 3 | 13 | 2 | 2.60 | 1.640 | 0.271 |
| 3 | 14 | 1 | 3.50 | 0.947 | 0.127 |
| 3 | 15 | 0 | 5.00 | 0.377 | 0.009 |
| | isopropyl acetate | | | | |
| 3 | 13 | 2 | 2.60 | 2.430 | 0.436 |
| 3 | 14 | 1 | 3.50 | 0.461 | 0.026 |
| 3 | 15 | 0 | 5.00 | 0.336 | 0.000 |
| | butyl acetate | | | | |
| 3 | 14 | 1 | 3.50 | >3.5 | >0.65 |
| 3 | 15 | 0 | 5.00 | 1.080 | 0.155 |
| | propyl/isopropyl | | | | |
| | acetate (1:3) | | | | |
| 3 | 13 | 2 | 2.60 | 1.626 | 0.268 |
| | propyl/isopropyl | | | | |
| | acetate (3:1) | | | | |
| 3 | 13 | 2 | 2.60 | 1.381 | 0.217 |
| | propyl/isopropyl | | | | |
| | acetate (1:1) | | | | |
| 3 | 13 | 2 | 2.60 | 1.135 | 0.166 |

The Table 1 data show several trends. First, within the homologous series of alkyl acetates, the propyl and isopropyl acetates clean the most contaminants from the contaminated glycol. Although all acetates precipitated HMWPC when used in a very high ratio (5:1), it appears that the lower and higher molecular weight members of the series are less effective. In addition, if the goal is to use a smaller amount of acetate to efficiently precipitate HMWPC, then the isopropyl acetate is less effective than propyl acetate. Isopropyl acetate shows a non-linear collapse of performance at a ratio of 2.6:1 TC/(DG+TEG) although it has demonstrably better performance at high ratios.

A second observation concerns various mixtures of alkyl acetates. When propyl acetate is mixed at various ratios with isopropyl acetate, the 1:1 blend performs much better than the 2:1 and 1:2 ratios. Although not shown in Table 1, the addition of butyl acetate even in small amounts causes a collapse of performance. Examining these mixtures at the 2.6:1 ratio of TC/(DG+TEG) clearly shows that a 1:1 blend of isopropyl and propyl acetate will provide a superior result with roughly 83% reduction in HMWPC contamination.

If the goal is to obtain extremely high purity of the glycol in a single pass, a ratio of greater than 3:1 acetate:glycol may be required, and a 1:1 volumetric blend of propyl to isopropyl acetate may be used. Reductions of >97% can be obtained using 3.5:1 ratio of alkyl acetate to contaminated glycol.

Without being limited by theory, it is believed that the tested alkyl acetates result in precipitation of the contaminants because while they are miscible with the glycol, their Hansen solubility parameters (HSP) are poorly matched to the glycol. For example, Table 2 shows the HSP values for the alkyl acetates listed above. It can be seen in Table 2 that the homologous series of acetates all have very similar HSP values, and that while they match the dispersion value of TEG, they are a poor match for the polar parameter and especially the hydrogen bonding parameter. It is believed that the HMWPC have a significantly elevated hydrogen bonding parameter, such that as the surrounding fluid is diluted with the alkyl acetates, the HMWPC collapses into a precipitate that can no longer be dissolved. It is also believed that the polarization parameter is the more important of the parameters, with a value of 4.3 to 5.3 being effective.

In view of the above, it is believed that other solvents that are miscible with glycol, and whose Hansen solubility parameters (HSP) are poorly matched to the glycol, will also be usable to clean contaminated glycol.

TABLE 2
HSP VALUES FOR GLYCOLS AND SOLVENTS

| CHEMICAL NAME | δ_{d} | δp | δh | Vm |
|---|---|---|---|---|
| Triethylene glycol | 16.0 | 12.5 | 18.6 | 114 |
| Diethylene glycol | 16.6 | 12.0 | 20.7 | 94.9 |
| Ethylene glycol | 17.0 | 11.0 | 26.0 | 55.8 |
| Methyl acetate | 15.5 | 7.2 | 7.6 | 79.7 |
| Ethyl acetate | 15.8 | 5.3 | 7.2 | 98.5 |
| Propyl acetate | 15.3 | 4.3 | 7.6 | 115.3 |
| Isopropyl acetate | 14.9 | 4.5 | 8.2 | 117.1 |
| Butyl acetate | 15.8 | 3.7 | 6.3 | 132.5 |

### Re-Dissolution of Contaminants

After filtering the precipitate from the mixture, it was attempted to re-dissolve the precipitate with water, triethylene glycol and ethylene glycol. This was unsuccessful. This is notable, as prior to precipitation, the contaminants fully dispersed/dissolved into water and ethylene glycol. It is believed that the precipitated HMWPC has either formed tight hydrogen bonds, cross linked, or has formed strong van der Waal bonds to form a complex that is no longer easily dissolved by nearly any solvent.

### Experiments

Two samples of triethylene glycol (TEG) were obtained from operating natural gas dehydration units. The first is called “Hanlon Lean” and the second is called “Gordon Lean”. Experiments were carried out to understand these two samples and to investigate improved methods of eliminating dark-colored contamination found in these materials.

Filtration was carried out with Whatman Grade 5 filter paper rated at approximately 2.5 μm. Reagents reported are Analytical Reagent (AR) grade. Acetate cleaning agent was a 1:1 mixture of isopropyl acetate and n-propyl acetate. Hydrogen peroxide was 35% by weight hydrogen peroxide H₂O₂. In much of the data reported herein, this is converted to a 100% pure basis weight.

The glycol samples were obtained from the field and pre-filtered through a Whatman Grade 5 filter prior to use, to reduce interference caused by particulate materials in the sample. Optical opacity of samples was analyzed using a Hach DR 5000 spectrophotometer operating in single wavelength mode and set at 640 nm. A 1.0″ square optical cuvette was used to hold samples while being analyzed. A hot plate used to process the glycol samples was a four-station system set to a surface temperature of approximately 220° C. Up to four 50-ml beakers were placed at each of the four stations. It was found that metal immersion thermocouples used to monitor solution temperature react with hydrogen peroxide at elevated temperature and interfere with the reaction. Therefore, the metal immersion thermocouples were used to monitor temperature only occasionally.

### Experiment A

A sample of Lean Hanlon was directly assayed for optical density by 10:1 dilution with clean AR-grade TEG. A reading of 2.001 was obtained for this diluted sample giving an implied optical density of the as-received, but filtered, sample of 20.01.

### Experiment B

20.07 grams filtered Lean Hanlon was mixed with 100.0 grams of an acetate cleaning solution. Following a time period (less than 5 minutes), the sample was filtered. The acetate is then boiled off to leave the original TEG. This was diluted 5:1 with clean TEG and has a measured optical density of 0.657 (3.285 accounting for dilution by clean TEG). This implies that the standard treatment of the sample with 5:1 volume ratio of acetate solution results in an 84% reduction in optical density.

### Experiment C

The impact of water on the precipitation of contamination from the Lean Hamlon sample was studied. 20 g of Lean Hamlon was mixed with 100 g of AR-grade deionized water. This mixture is filtered and no precipitate on the filter paper was observed. Next, this sample was boiled to drive off the moisture and obtain a glycol with 37.52 AU optical opacity. This is an increased value as compared to the original sample, which indicates the creation of an optically-active contaminant. A combination of 20 g Lean Hamlon, 50 g acetate solution and 50 g deionized water was boiled to drive off both the acetate and the water. The remaining glycol was filtered and the optical density of the sample was observed to be 19.83 AU. Precipitate contamination from a water-loaded sample was not achieved and as such, the presence of water may not be desirable.

FIG. 2 illustrates the addition of small amounts of water to glycol which results in the formation of a two-phase system when mixed with acetate cleaning solution, indicating that it may be desirable to remove moisture prior to processing.

### Experiment D

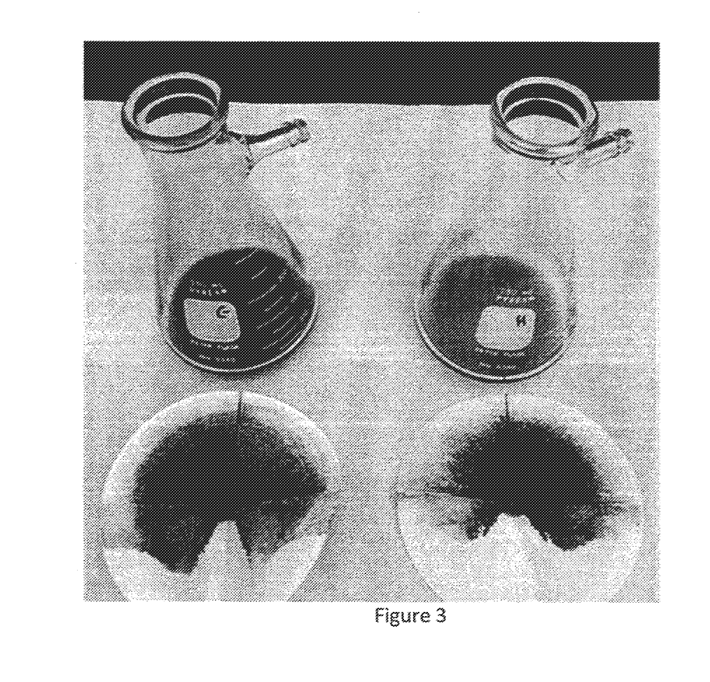

Each of four 50-ml beakers contained 5 g of filtered Lean Hamlon and 0.07 g, 0.10 g, 0.15 g, and 0.20 g of deionized water was added. 25 g of acetate cleaning solution was added and the beakers were stirred. With increasing amounts of trace water, it was observed that a distinct phase separation occurred with a dark TEG layer at the bottom of each beaker and a light, honey-colored, acetate layer above. These layers were filtered and it was observed that as water increased, the amount of dirt collected on the filter was reduced (see FIG. 3).

### Experiment E

Three 50-ml beakers contained 10 g of Lean Gordon sample to each beaker along with i) 0.20 g 40% by weight glyoxal solution to beaker 1, ii) 0.20 g 35% hydrogen peroxide to beaker 2, and iii) 0.03 g copper acetate and 0.20 g 35% hydrogen peroxide. The beakers were placed on a hot plate for 15 minutes and the formation of bubbles on those samples containing hydrogen peroxide was observed, while the glyoxal-treated sample appeared to be quiet. The beakers were removed from the hot plate and allowed to cool. 25 g of acetate solution was added to each beaker. These were mixed and after a brief interval, the samples were filtered. The measured optical density of the glyoxal sample was >3.5 AU (too high to read), 0.652 AU for the copper-peroxide mixture, and 0.346 AU for the peroxide sample. For comparison, a control that was heated, acetate treated and then filtered was 1.368 AU. From this, a standard cross-linking agent, such as glyoxal was not effective. From this, a metal such as divalent copper is not a catalyst that further enhances the performance of the hydrogen peroxide treatment.

FIG. 3 illustrates glyoxal (left) and peroxide (right) samples after treatment from Experiment E.

### Experiment F

To examine the dose-response curve for treating contaminated glycol samples with hydrogen peroxide, 10 g of the Lean Gordon sample was loaded into each of three 50-ml beakers. Varying amounts of H₂O₂ was added into the glycol and the mixture was heated for 8 minutes on a hot plate to a temperature of 161° C. at the end of the 8 minutes. The samples were allowed to cool and 25 g of acetate cleaning solution was added to each beaker. The final results are shown in Table 3. The original optical density of filtered Lean Gordon sample is 21.00 AU.

TABLE 3
Lean Gordon Response to Peroxide and Acetate Treatment

| Sample | H₂O₂ Added | Optical Density | Dilution-Corrected AU |
|---|---|---|---|
| 1 | 0.000 control | 1.368 | 6.840 (67.4% reduction) |
| 2 | 0.047 (0.165%) | 0.981 | 4.905 (76.6% reduction) |
| 3 | 0.109 (0.382%) | 0.455 | 2.275 (89.2% reduction) |
| 4 | 0.200 (0.700%) | 0.346 | 1.730 (91.8% reduction) |
| 5 | 0.330 (1.155%) | 0.101 | 0.505 (97.6% reduction) |

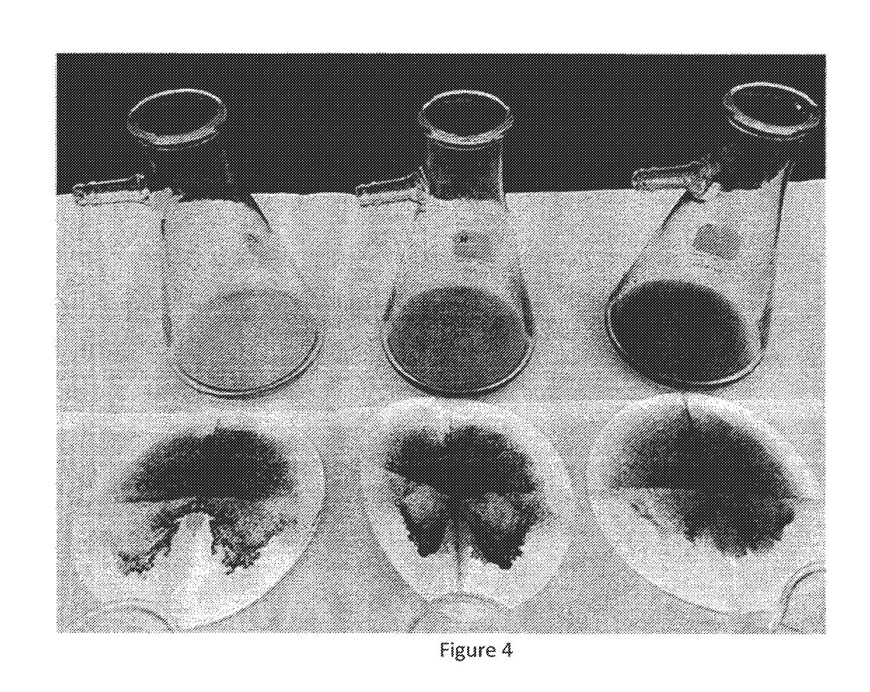

The data shown in Table 3 indicates a smooth trend of improved contamination reduction at progressively higher hydrogen peroxide treatment (as shown in FIG. 4).

FIG. 4 illustrates filtrates and filters of 10-g samples of glycol first treated with 0.330, 0.109 and 0.047 g H₂O₂ followed by a 2.5:1 weight of acetate solution. Results indicate that H₂O₂ may enhance the cleaning process.

### Experiments G and H

A variety of experiments were carried out with a combination of hydrogen peroxide and acid or base. The acid used was concentrated hydrochloric acid and the base was concentrated ammonium hydroxide. These were selected because they have properties that are volatile and will be vaporized along with water and any residual hydrogen peroxide during the treatment process. Regardless of the concentrations tested, the use of high or low pH conditions did not improve, and in some cases materially reduced the effectiveness of the hydrogen peroxide treatment. It was, therefore, concluded that roughly neutral conditions of the as-received sample were used for carrying out the hydrogen peroxide treatment process.

### Experiment I

12 g of filtered Lean Hanlon sample was loaded into a set of 50-ml beakers. The samples were heated on a hot plate for approximately 10 minutes to a temperature of 150° C. The samples were then allowed to cool and 12 g of acetate solution was added to each sample. The samples were allowed to sit for 20 minutes (samples 1-8) or 50 minutes (samples 9-12) to provide time for the precipitation of the contaminant. In the first series (samples 1-8), the amount of H₂O₂ used progressively increase during the treatment and a constant amount of acetate solution (in a 1:1 amount against the weight of TEG) was used. In the second series of tests (samples 9-12), the amount of H₂O₂ was constant at 2.92% by weight, but the acetate applied after H₂O₂ treatment was systematically varied. Table 4 summarizes the results. The formation of a coarse black-colored precipitate may take time and may be slower in samples receiving a more aggressive treatment with H₂O₂ and slower for samples treated with smaller amounts of acetate solution.

TABLE 4
Lean Hanlon Response to Peroxide and Acetate Treatment

| | | | Optical | Dilution- |
|---|---|---|---|---|
| Sample | H₂O₂ (g) | Acetate (g) | Density | Corrected AU |
| 1 | 0.16 (0.47%) | 12.0 | 2.568 | 5.136 (74.3%) |
| 2 | 0.24 (0.70%) | 12.0 | 1.844 | 3.688 (81.6%) |
| 3 | 0.36 (1.05%) | 12.0 | 1.602 | 3.204 (84.0%) |
| 4 | 0.51 (1.49%) | 12.0 | 1.132 | 2.264 (88.7%) |
| 5 | 0.51 (1.49%) | 12.0 | 1.189 | 2.378 (88.1%) |
| 6 | 0.66 (1.93%) | 12.0 | 0.980 | 1.960 (90.2%) |
| 7 | 0.86 (2.51%) | 12.0 | 0.892 | 1.784 (91.1%) |
| 8 | 1.03 (3.00%) | 12.0 | 0.594 | 1.188 (94.1%) |
| 9 | 1.00 (2.92%) | 12.0 | 0.701 | 1.402 (93.0%) |
| 10 | 1.00 (2.92%) | 10.0 | 0.897 | 1.644 (91.8%) |
| 11 | 1.00 (2.92%) | 8.0 | 1.224 | 2.040 (89.8%) |
| 12 | 1.00 (2.92%) | 6.0 | 1.721 | 2.582 (87.1%) |

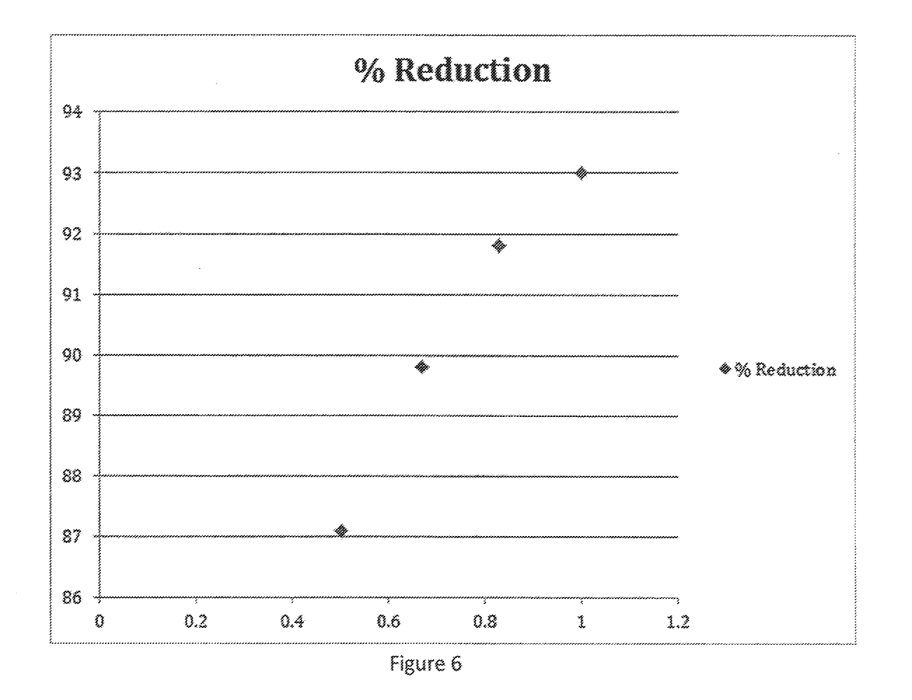

The data in Table 4 and FIG. 7 show that even small amounts of H₂O₂ are able to provide removal of contaminant from glycol when an adequate amount of acetate cleaning solution is later applied. As amounts of H₂O₂ increase, the performance increases from roughly 74.3% decontamination at 0.47% H₂O₂ by weight to 94.1% contaminant reduction at 3.00% of applied H₂O₂. As hydrogen peroxide in these amounts is of low cost, a higher concentration (2.92% by weight) of H₂O₂ was used and examined in the second series (samples 9-12) to explore how this might reduce the need for acetate solution. At elevated use of H₂O₂, acetate can be reduced to as low as about 0.5:1 acetate:glycol ratio, while sustaining 87.1% contaminant reduction. Without H₂O₂ treatment, the application of 1:1 ratio of acetate:glycol produced <50% contaminant reduction, while it increased to 93% with H₂O₂ treatment and 1:1 acetate cleaning (see FIG. 6).

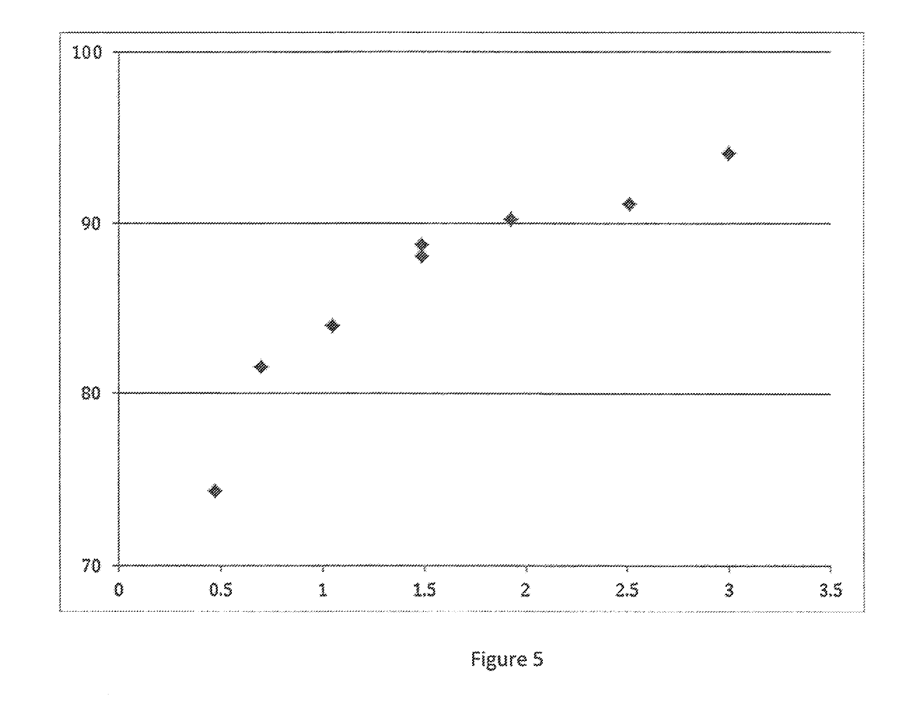

FIG. 5 illustrates a plot of percent reduction in optical density of a sample (dilution corrected) vs. amount of applied H₂O₂ used in a treatment process. Although the use of 3% by weight H₂O₂ provided improvements, modest amounts of H₂O₂ further increased performance. Without the treatment, the samples would have shown <<50% reduction.

FIG. 6 illustrates a percent reduction in contaminant (optical density) on Y-axis vs. acetate:glycol ratio. At a fixed 2.92% by weight of hydrogen peroxide treatment, smaller amounts of acetate can be used to obtain a suitable degree of contaminant reduction. Whereas an acetate:glycol ratio of 5:1 without hydrogen peroxide treatment is used to obtain perhaps 84% contaminant reduction (Experiment B), this can be reduced to 0.5:1 (ten-times less) when hydrogen peroxide treatment is used. An increase in acetate:glycol ratio to 1:1 results in advantageous deep cleaning in the range of 93% for a single pass.

While the above description provides examples of one or more processes or apparatuses, it will be appreciated that other processes or apparatuses may be within the scope of the accompanying claims.

1. A process for cleaning contaminants from a contaminated fluid, the process comprising:
a) combining the contaminated fluid with a less polar miscible solvent to yield a reduced polarity contaminated fluid-solvent mixture in which the contaminants are insoluble, at least a portion of the contaminants precipitating from the reduced polarity contaminated fluid-solvent mixture to yield precipitated contaminants and the reduced polarity contaminated fluid-solvent mixture;
b) separating at least a portion of the precipitated contaminants from the reduced polarity contaminated fluid-solvent mixture; and
c) separating at least a portion of the fluid from the reduced polarity contaminated fluid-solvent mixture to yield cleaned fluid.

2. The process of claim 1 further comprising:
d) prior to combining the contaminated fluid with the less polar miscible solvent, combining hydrogen peroxide with the contaminated fluid and heating the hydrogen peroxide-contaminated fluid mixture.

3. The process of claim 1, wherein the contaminated fluid comprises an amine fluid or a glycol selected from the group consisting of triethylene glycol, diethylene glycol, tetraethylene glycol, ethylene glycol, and combinations thereof.

4. The process of claim 1, wherein the contaminated fluid comprises contaminated triethylene glycol.

5. The process of claim 1, wherein the less polar miscible solvent comprises an alkyl acetate.

6. The process of claim 5, wherein the alkyl acetate is selected from the group consisting of methyl acetate, ethyl acetate, propyl acetate, isopropyl acetate, butyl acetate, and combinations thereof.

7. The process of claim 1, wherein the less polar miscible solvent comprises a mixture of propyl acetate and isopropyl acetate.

8. (canceled)

9. The process of claim 1, wherein the process is one of a continuous process, a semi-continuous process, a batch process.

10. The process of claim 1, wherein step b) comprises filtering the precipitated contaminants from the reduced polarity glycol-solvent mixture.

11. The process of claim 1, wherein the contaminants comprise high molecular weight polar contaminants.

12. (canceled)

13. The process of claim 1, wherein step c) comprises evaporating the less polar miscible solvent from the reduced polarity glycol-solvent mixture.

14. (canceled)

15. The process of claim 1 wherein the contaminated fluid is a contaminated glycol, and wherein the contaminated glycol and the less polar miscible solvent are combined at a volumetric ratio of between about 1:0.5 and about 1:5.

16. (canceled)

17. A process for precipitating contaminants from contaminated glycol, the process comprising:
combining the contaminated glycol with a less polar miscible solvent to yield a reduced polarity glycol-solvent mixture in which the contaminants are insoluble, at least a portion of the contaminants precipitating from the reduced polarity glycol-solvent mixture to yield precipitated contaminants and the reduced polarity glycol-solvent mixture.

18. The process of claim 17 further comprising:
prior to combining the contaminated glycol with the less polar miscible solvent, combining hydrogen peroxide with the contaminated glycol and heating the hydrogen peroxide-glycol mixture.

19. The process of claim 17, wherein the contaminated glycol comprises a glycol selected from the group consisting of triethylene glycol, diethylene glycol, ethylene glycol, tetraethylene glycol, and combinations thereof.

20. The process of claim 17, wherein the contaminated glycol comprises contaminated triethylene glycol.

21. The process of claim 17, wherein the less polar miscible solvent comprises an alkyl acetate.

22. The process of claim 17, wherein the alkyl acetate is selected from the group consisting of methyl acetate, ethyl acetate, propyl acetate, isopropyl acetate, butyl acetate, and combinations thereof.

23-27. (canceled)

28. A process for cleaning high molecular weight polar contaminants from contaminated triethylene glycol, the process comprising:
a) combining the contaminated triethylene glycol with an alkyl acetate solvent to yield a reduced polarity triethylene glycol-alkyl acetate mixture in which the contaminants are insoluble, at least a portion of the high molecular weight polar contaminants precipitating from the reduced polarity triethylene glycol-alkyl acetate mixture to yield precipitated high molecular weight polar contaminants and the reduced polarity triethylene glycol-alkyl acetate mixture;
b) separating at least a portion of the precipitated high molecular weight polar contaminants from the reduced polarity triethylene glycol-alkyl acetate mixture; and
c) separating at least a portion of the triethylene glycol from the reduced polarity triethylene glycol-alkyl acetate mixture.

29. The process of claim 28 further comprising:
prior to combining the contaminated triethylene glycol with the alkyl acetate solvent, combining hydrogen peroxide with the contaminated glycol and heating the hydrogen peroxide-glycol mixture.

30-36. (canceled)
